(19) United States
(12) Patent Application Publication
(10) Pub. No.: US 2023/0147529 A1
(43) Pub. Date: May 11, 2023

(54) PREPARATION OF A NEEDLE SENSOR FOR A BODY MONITORING SYSTEM

(71) Applicant: PKvitality, Paris (FR)

(72) Inventors: Kevin LY HAK, VILLIERS SUR MARNE (FR); Florian BADIOLA, PARIS (FR); Luc PIERART, VILLEJUIF (FR)

(73) Assignee: PKvitality, Paris (FR)

(21) Appl. No.: 17/996,158

(22) PCT Filed: Apr. 16, 2021
(86) PCT No.: PCT/FR2021/050677

§ 371 Date: Oct. 13, 2022

(30) Foreign Application Priority Data
Apr. 16, 2020 (FR) FR2003820

(51) Int. Cl. A61B 5/1495; A61B 5/145; A61B 5/00; A61B 5/1473

(52) U.S. Cl. CPC A61B 5/1495; A61B 5/14532; A61B 5/14514; A61B 5/685; A61B 5/681; A61B 5/1473; A61B 2560/0238; A61B 2560/0228; A61B 2560/0456

(57) ABSTRACT

The present invention relates to a sensor preparation assembly (30) for a body monitoring system, the sensor (30) comprising at least one needle, the preparation assembly comprising: a bag (26) comprising a volume of a preparation solution, the bag (26) being configured to be positioned under the sensor (30) and being configured to be pierced by the needle (32), a receptacle (28) on which the bag is arranged (26), bearing means (24) that arc movable in relation to the receptacle (28) toward a bearing position, the bearing means (24) in the bearing position being configured to cause the sensor (30) to pierce the bag (26). The invention further relates to a sensor preparation method, comprising the placement of the sensor (30) in a position that is interposed between the bearing means (24) and the bag (26), the needle (32) of the sensor (30) piercing the bag (26).

### FIELD OF THE INVENTION

The present invention belongs to the technical field of wearable devices used in body monitoring systems, for example for reading and monitoring biochemical parameters of the human body. The invention relates to a needle sensor preparation assembly for a body monitoring system, as well as a sensor kit comprising such an assembly. The invention further relates to a method for preparing a needle sensor. The invention is in particular of advantageous application for the calibration of a needle sensor, preferably before its integration into a wearable device.

### PRIOR ART

The monitoring of many known chronic diseases in humans requires daily reading of biochemical parameters. A concentration level of a body analyte in a body fluid of the body, for example in blood plasma or in the interstitial fluid of body cells, may be recorded.

As a common example, monitoring diabetes in a patient requires an accurate daily reading of the patient's glycaemia.

A common solution for monitoring diabetes consists in carrying out a puncture, for example at the end of the finger, to cause a drop of blood to bead up, then in carrying out a daily measurement of glycaemia in the drop of blood thus obtained.

Monitoring systems have been proposed to dispense with the need for a manual puncture, so as to make glycaemia measurement less laborious and less invasive. This is called CGM for “Continuous Glucose Monitoring” systems. Some of these CGM systems carry out, at regular intervals, a glycaemia measurement at the interstitial liquid between the cells of the skin. Interstitial fluid glycaemia is very close to blood plasma glycaemia. Measurements at the interstitial fluid allow a simple and minimally invasive follow-up of the glycaemia of the patients; these measurements can be carried out using needle sensors, transcutaneously, or non-invasively, such as for example by iontophoresis or implantably with a measurement by chemo-fluorescence.

The international application published under the number WO 2018/104647 describes a body monitoring system, which can be used in particular for monitoring glycaemia. This monitoring system includes an electronic watch that can be attached to the wrist using a bracelet. The watch has a case, in which is inserted an interchangeable removable capsule containing a micro-needle sensor. The sensor is automatically controlled by the electronics of the case, to perform a transcutaneous measurement. The glycaemia measurement by the sensor is an electrochemical measurement.

The body monitoring system described in the aforementioned document has the significant advantage of providing an autonomous measurement of calibration by measuring a reference concentration of a body analyte in the user.

The number of manipulations that the user must perform to obtain his daily measurements is greatly reduced. In particular, the user needs to perform few manual punctures (for example a simple weekly puncture), or even no more manual punctures. Another advantage of this system is its low hygienic risk, because the sensor needles are not in contact with the external environment, once these needles are inserted into the skin.

In addition, the maintenance of the aforementioned system is simple, since to replace a faulty sensor, it is sufficient to remove the removable capsule and insert a new capsule.

However, a significant amount of time—for example several months—may elapse between the production of the removable capsule including the micro-needle sensor and the insertion of the removable capsule into a wearable device worn by the user. It has been observed that the quality of the glycaemia measurement provided by the sensor deteriorates during such a long period of non-use of the sensor.

The deterioration of the measurement performance of the needle sensor is explained by a drift in the electrochemical measurement over time. This drift may be due to an unsuitable ambient temperature in the storage areas of the removable capsules comprising the needle sensors. In particular, excessively hot temperatures are detrimental to the integrity of the measurement system. If sudden and significant variations in the temperature of the sensor occur, this aggravates the drift of the electrochemical measurement.

A first known method to overcome this drift is a “factory” calibration carried out on the scale of an entire batch of needle sensors, after the production of the batch. An alphanumeric code is then written on a packaging of the removable capsule containing the sensor, or is integrated into an electronic memory of the sensor. The wearable device has an abacus in which each alphanumeric code is associated with a reference value. When inserting the capsule into the wearable device, the alphanumeric code is read and calibration can be performed.

This factory calibration known to the prior art, based on the use of charts, does not, however, give complete satisfaction. Drifts in the electrochemical measurement provided by the sensor are always observed.

A second known calibration method, sometimes used in combination with the first one, consists of calibration measurements (manual punctures) performed regularly by the user while wearing the wearable device. The calibration measurements are carried out manually by the user, for example on a daily or weekly basis. To perform manual calibration, the user may need to enter a capillary glycaemia value on the wearable device.

In this second method, which can be combined with the first method, the user is responsible for the correct calibration of his own wearable device. Regular manual operation is required, which defeats one of the purposes of an autonomous body monitoring system, namely to limit the discomfort felt by the user.

In view of the above considerations, the prior art does not give satisfaction concerning the preparation of needle sensors for wearable devices, such as electronic watches. Some known systems do not ensure lasting precision of the electrochemical measurement, and other known systems require regular calibrations which must be implemented by the user.

The prior art does not give satisfaction either concerning the speed of commissioning of needle sensors, when using a new sensor. The convergence of the electrochemical measurement provided by the sensor after insertion of the needle into the human interstitial fluid, that is to say the achievement of an equilibrium between the chemical species of the sensor allowing the measurement and the medium, is currently quite slow. The convergence time of the known sensors is for example several hours. During this commissioning time, the measurement provided is not reliable, and the user runs a risk because he is not informed of the fluctuations of his biochemical parameters.

Moreover, document US 2005/130292 describes a biochip integrating a calibration solution pouch. However, the calibration solution pouch is not designed to be positioned under the sensor or pierced directly by a sensor needle. The calibration solution described in this document is not used to calibrate a sensor needle involved in an electrochemical measurement.

### GENERAL DESCRIPTION OF THE INVENTION

An objective of the invention is to propose improvements for a body monitoring system comprising a needle sensor, so that the measurement provided by this sensor remains reliable and precise over time.

In particular, a solution to easily perform the calibration of this sensor, while limiting the inconvenience caused to the user is sought. The objective is to eliminate the need for daily manual punctures and to switch to at least weekly manual punctures, or even to eliminate the need for manual punctures.

As a corollary, it is desired that the user of the body monitoring system is not responsible for the correct calibration of said sensor, during the life cycle of the monitoring system. It is desired to avoid the need to regularly puncture the finger to carry out a daily calibration of the sensor.

The solution sought must remain very easy to use, in order to ensure a pleasant user experience.

A secondary objective is to allow the rapid commissioning of a new needle sensor, after insertion of this sensor into a wearable device. For this purpose, it is necessary to accelerate the convergence of the electrochemical measurement between the chemical compounds of the sensor and the medium into which the needle of the sensor is inserted, typically the human interstitial liquid. Moreover, it is desired to retain a simple body monitoring system, with a low cost price, compatible with large-scale industrialization.

As such, the invention relates, according to a first aspect, to a sensor preparation assembly for a body monitoring system, the sensor comprising at least one needle, the preparation assembly comprising:

a pouch comprising a volume of a preparation solution, the pouch being configured to be positioned under the sensor and being configured to be pierced by the needle,

a receptacle on which the pouch is arranged,

bearing means that are movable in relation to the receptacle toward a bearing position, the bearing means in the bearing position being configured to cause the sensor to pierce the pouch.

In a sensor preparation assembly according to the invention, the preparation solution contained in the pouch allows to easily and quickly prepare the needle sensor. It is not necessary, for a user of the body monitoring system wishing to prepare the sensor, to perform a finger puncture or to directly handle the needles of the sensor in any way.

The preparation assembly of the invention is used for example when commissioning the needle sensor. During the first use of a wearable device integrating the sensor, or during the commissioning of a new sensor, the user can easily carry out the preparation of the sensor himself by using the bearing means to pierce the pouch with the sensor needle.

The preparation of the sensor is for example a pre-calibration of the sensor, in order to correct a possible drift of an electrochemical measurement provided by the sensor. This operation can be called “pre-calibration”, because it takes place at the start of the use cycle of the sensor.

The preparation of the sensor is, as an alternative to the pre-calibration or in combination, a pre-wetting of the needle(s) of the sensor.

The sensor preparation assembly according to the first aspect of the invention may have, optionally and without limitation, the following technical characteristics taken alone or in any of the technically possible combinations:

the preparation solution is a calibration solution having a concentration by volume of a body analyte equal to a predetermined reference value,

the body analyte is glucose,

the predetermined reference value is comprised between 2 millimoles of glucose per liter and 20 millimoles of glucose per liter, preferably between 6 millimoles of glucose per liter and 10 millimoles of glucose per liter,

the preparation solution is a pre-wetting solution,

in the bearing position, the bearing means cover the sensor,

the preparation assembly further comprises an electronic apparatus, the electronic apparatus preferably being configured to control a concentration measurement of a body analyte via the sensor, and/or being configured to generate an electric current,

the electronic apparatus comprises a wearable device, preferably an electronic watch or bracelet or a tracker,

the electronic apparatus comprises a processing unit and a memory, configured to record and manage information on the resetting of a measurement of the body analyte concentration,

part of the electronic apparatus forms the bearing means or is part of the bearing means,

the bearing means comprise a cover, the cover preferably being configured to completely cover the bearing means,

the preparation assembly comprises a box, at least part of the bearing means being integrated into the box,

the pouch and the receptacle are received in the box,

the box comprises at least one housing configured to stably arrange the receptacle in the box, and/or configured to stably arrange a wearable device in the box,

the bearing means comprise at least one mechanical clip configured to perform a bearing in Z on the sensor, or on part of an electronic apparatus,

the preparation assembly further comprises a peelable film, the peelable film preferably forming with the receptacle an enclosure which encloses the pouch and/or which encloses the sensor,

the preparation assembly further comprises a preparation station configured to receive the pouch,

the preparation station comprises an indicator to inform a user of the duration of maintaining the bearing position,

the electronic apparatus comprises an indicator to inform a user of a duration of maintaining the bearing position,

the preparation station is configured to charge a battery of an electronic apparatus comprised in the preparation assembly,

the preparation station further comprises at least one bearing cover and/or comprises two mechanical clips movable relative to each other in a separation direction,

at least one mechanical clip comprises an electrical charging contact.

According to a second aspect, the invention relates to a sensor kit including a preparation assembly as defined above, the sensor kit further including a sensor comprising at least one needle, the sensor being configured so that, in the bearing position of the bearing means, the needle is partially or totally immersed in the preparation solution.

Optionally and without limitation, the sensor further comprises a memory configured to record information on the resetting of a concentration measurement of a body analyte.

In the latter case, an advantage is that the need to record the resetting information in a device separate from the sensor during a calibration is eliminated.

According to a third aspect, the invention relates to a method for preparing a sensor for a body monitoring system, the method being implemented using a sensor kit as defined above, the method comprising a step of placing the sensor in a position that is interposed between the bearing means and the pouch, the bearing means being placed in the bearing position so that the needle of the sensor pierces the pouch.

The preparation method according to the third aspect of the invention may have, optionally and without limitation, the following technical characteristics taken alone or in any of the technically possible combinations:

the bearing means are integrated into an electronic apparatus which further comprises a processing unit,

the processing unit is used to control the needle sensor such that the needle sensor acquires a concentration measurement of a body analyte, and/or the processing unit is used to generate an electric current in the sensor,

the method comprises additional steps of:

acquiring by the sensor a measurement of the concentration of a body analyte in the solution for preparing the pouch;
transmitting the measurement to the processing unit;
and calculating a resetting signal according to the measurement, so as to calibrate the sensor,

a wearable device is interposed between the bearing means and the sensor,

the bearing means comprise a cover which presses on the wearable device,

the bearing means are maintained in the bearing position for a duration comprised between 10 seconds and 2 hours, in order to press the sensor,

at the end of the preparation of the sensor, the pouch is discarded, and a new pouch is used during the preparation of a separate sensor.

### GENERAL DESCRIPTION OF FIGURES

Other characteristics, objects and advantages of the invention will emerge from the description which follows, which is purely illustrative and not limiting, and which must be read in conjunction with the appended drawings, among which:

FIG. 1a is a schematic illustration of the different elements of a sensor kit, the sensor kit comprising an electronic watch.

FIG. 1b is a schematic front view of the electronic watch shown in FIG. 1a.

FIG. 2 is a perspective top view of a needle sensor according to an example. This sensor can be used in conjunction with any one of the sensor preparation assemblies shown in the appended figures.

FIG. 3 shows a sensor preparation assembly according to a first embodiment, comprising a box.

FIG. 4 shows a station for charging an electronic watch, said charging station being able to be integrated into a sensor preparation assembly according to a second embodiment.

FIG. 5a is a cross-sectional view of the charging station of FIG. 4, with the clips of the charging station in the position close to each other.

FIG. 5b is a cross-sectional view of the charging station of FIG. 4, with the clips of the charging station in the position separated from each other.

FIG. 6 illustrates steps of a sensor preparation method.

### DETAILED DESCRIPTION OF EMBODIMENTS OF THE INVENTION

The description below relates to the preparation of a sensor with needle(s) integrated into an electronic watch. The sensor is designed to provide a measurement of glucose concentration within the interstitial fluid of a wearer. The electronic watch constitutes with the sensor a body monitoring system.

“Body monitoring” means the verification of biochemical constants of the wearer of the monitoring system, typically the concentration of the interstitial liquid of the wearer in a protein, a hormone, a biomarker, in oxygen, in nutrients, etc. The person skilled in the art will easily understand that other physical quantities can be monitored by the monitoring system, such as, for example, lactate concentration, hydration, etc.

In what follows, the biochemical constant to be monitored is the concentration of glucose (or glycaemia) within the interstitial liquid of the skin. Interstitial fluid glycaemia is considered representative of blood plasma glycaemia. It will be understood that the sensor preparation assembly described below can be used, with the same advantages, to prepare a sensor intended to measure another biochemical parameter. Furthermore, the needle or needles of the sensor could be intended to be inserted into a body fluid other than the interstitial fluid, for example into the blood.

Furthermore, the wearable device is, throughout the description below, an electronic watch configured to display information for the attention of its wearer. However, the sensor preparation assembly described below can be used, with the same advantages, in combination with any other type of wearable device: bracelet, tracker, etc. In all of the appended figures and throughout the description below, similar elements bear identical reference numerals.

### General Architecture of a Sensor Preparation Assembly

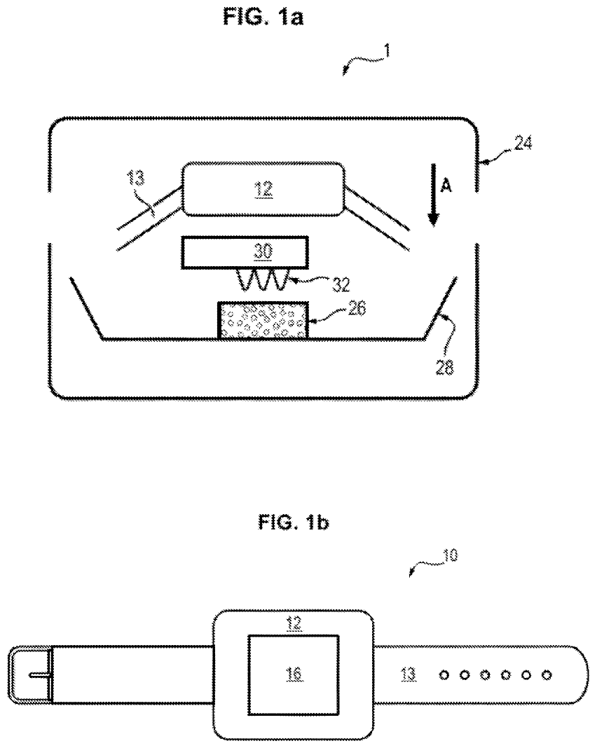

FIG. 1a schematically shows a sensor kit 1 according to an example. The kit 1 comprises a sensor 30 intended for the regular monitoring of an individual's glycaemia. The sensor 30 is intended to provide a measurement of electric current within the interstitial liquid. The kit 1 also comprises a sensor preparation assembly.

In the present example, the preparation assembly comprises in particular an electronic watch 10, intended to be worn on the wrist of the wearer.

The preparation assembly includes bearing means 24, a pouch 26 and a receptacle 28, examples of the structure of which will be described below.

The pouch 26 comprises a volume of a sensor preparation solution.

In the present example, the preparation solution is a calibration solution, having a concentration by volume of a body analyte equal to a predetermined reference value.

It is recalled that, in the present example, the body analyte to be analyzed is glucose, and the measurement provided by the sensor 30 is representative of glycaemia.

Thus, the solution contained in the pouch 26 has here a precisely known glucose concentration.

This concentration is advantageously comprised between 2 millimoles of glucose per liter and 20 millimoles of glucose per liter, more preferably between 6 millimoles of glucose per liter and 10 millimoles of glucose per liter.

Alternatively, the preparation solution contained in the pouch 26 can be a pre-wetting solution, that is to say a solution which reproduces the chemical nature of the body fluid in which the sensor 30 is intended to be inserted (namely the interstitial fluid here). The pre-wetting consists in moistening part of the chemical or biochemical materials (such as active enzymatic elements) which allow the electrochemical measurement, contained for example on the upper layers of the needles 32. These chemical elements typically include active enzymatic elements. Thanks to the pre-wetting, the subsequent passage of the electrical elements between all the chemical layers and the human body fluid (for example in the interstitial liquid), once the needle sensor is in place, is accelerated.

An advantage of carrying out a pre-wetting of the sensor 30, during a preparation of the sensor 30, is to accelerate the convergence of the subsequent measurements of the sensor in the body fluid. The sensor thus quickly reaches an operational state.

The preparation solution is produced here in the form of a gel.

Alternatively, the preparation solution can be a liquid solution.

It will be noted that the pouch 26 can contain a solution which can be used both, among other possible uses, as a calibration solution and as a pre-wetting solution.

Regardless of the form chosen for the preparation solution contained in the pouch 26, the pouch preferably further comprises an envelope which surrounds the volume of preparation solution, to allow the transport and handling of the pouch 26.

The pouch 26 can be pierced, so that a needle 32 of the sensor 30 can reach the preparation solution. If an envelope is present, it can be pierced at least partly.

The pouch 26 is arranged on one surface of the receptacle 28. The surface on which the pouch 26 is arranged is preferably stiff enough to oppose a force of resistance to the needle penetration. Thus, a needle pressed in the direction of the receptacle 28 can pierce the pouch 26.

For example, the pouch 26 is arranged on a flat surface of the receptacle 28.

The receptacle 28 has for example the shape of a cupola, with a flat surface for receiving the pouch 26 and one or more edges allowing to grip the cupola.

The pouch 26 can here be separated from the receptacle 28. However, the receptacle 28 can alternatively be secured to the pouch 26.

Furthermore, the sensor preparation assembly included in the kit 1 comprises bearing means 24 which are used to press the sensor 30, so that the sensor 30 pierces the pouch 26. The needles 32 of the sensor 30 are at the least partially immersed in the volume of preparation solution.

In the present example, the bearing means 24 include the watch 10 and include a cover which presses on the electronic watch. Thus, in FIG. 1a, the case of the electronic watch is interposed between the cover and the sensor.

It will be noted that the bearing means could comprise only a cover, configured to press the sensor 30 against the receptacle 28.

Alternatively, the bearing means could comprise only the watch.

The bearing means 24 are movable in relation to the receptacle 28, from a relaxed position to a bearing position, in a direction A.

In the released position, the sensor area which carries the needle 32 is remote from the pouch 26. The needle 32 does not pierce the pouch 26 in the released position of the bearing means. In the bearing position of the bearing means, the sensor area which carries the needle 32 is closer to the pouch 26, so that the needle 32 pierces the pouch 26. The bearing means 24 in the bearing position preferably cover the sensor 30.

To be brought from their relaxed position to their bearing position, the bearing means 24 can be handled by the user, for example with the hand, and/or be actuated by an additional device which can be an electronic device.

The bearing means in the bearing position can then be returned to the released position. The pressure of the sensor 30 on the pouch 26 is then released. Advantageously, the bearing means can be biased in a direction opposite to direction A by return means.

FIG. 1a schematically illustrates a state of the kit 1 in which the bearing means are in the released position. The needles 32 are distant from the pouch 26. In FIG. 1a, the released position corresponds to a high position of the bearing means and of the sensor. According to the orientation of this figure, a bearing position of the bearing means 24 corresponds to a lower position of the bearing means and of the sensor.

The bearing means 24 are preferably separated from the receptacle 28 and the pouch 26. In addition, if the bearing means 24 include a cover as in the kit 1 of FIG. 1a, said cover and the receptacle 28 have preferably complementary shapes. Thus, when the cover is pressed against the receptacle, the sensor 30 easily pierces the pouch 26 without the need for additional handling.

In one possible embodiment, the sensor preparation assembly comprises a film that covers the pouch 26 and/or that covers the sensor 30. The film is preferably arranged over the receptacle 28, and is typically attached to edges of the receptacle 28.

The film can thus delimit, with the receptacle 28, a packaging or “sterile pack” which contains the pouch 26 and/or the sensor 30. Such a film is not illustrated in FIG. 1a. An advantage of this construction is that the pouch 26 and the sensor 30 are separated from the external environment until they are used, and are easily stored and transported.

Said film is preferably a peelable film. Thus, the user wishing to prepare the sensor 30 can easily remove the peelable film to expose the pouch 26, and then prepare the sensor 30.

Optionally, the sensor kit 1 further comprises a base. The receptacle 28 is itself placed in the base. Here, the base has dimensions close to those of the cover. The base and the cover therefore together form a box.

It will be noted that, in a possible variant, the kit 1 does not include means specifically dedicated to the bearing of the sensor. The user then uses his hand directly to bring the needles 32 inside the pouch 26, by pressure in the direction A.

### Electronic Watch

FIG. 1b schematically shows a watch 10 according to an example.

The watch 10 in particular comprises a case 12, a bracelet 13 and a screen 16.

The watch 10 constitutes a wearable device, thanks to the bracelet 13 which allows it to be attached to the wrist of the user. The bracelet 13 is preferably adjustable.

The watch 10 is an electronic watch. The case 12 embeds the electronics necessary for the operation of the watch, in particular for the control of the operations relating to the monitoring of the glycaemia of the user of the watch. As such, the watch 10 comprises a processing unit 14.

The processing unit 14 comprises for example one or more processors contained in the case 12, and in particular allows to manage the information of acquisition of the body analyte concentration measurement.

The processing unit 14 is intended to be connected to the sensor 30, once the sensor 30 has been inserted into the watch 10. Computer code instructions for controlling the sensor 30 are pre-recorded in the processing unit 14. The watch 10 is thus configured to acquire a glucose concentration measurement, using the sensor 30. The watch 10 further preferably comprises a memory configured to save the glucose concentration measurements thus acquired.

The processing unit 14 is further configured to control the display of information on the screen 16. The information displayed preferably comprises time information and/or glycaemia monitoring information.

The watch 10 may in particular have a structure conforming to the watch described in document WO 2018/104647. Such a watch includes two modules that can be separated from each other; a first module includes the body of the case attached to the bracelet and the control electronics, and a second module is formed by an interchangeable removable capsule comprising the needle sensor. The two modules have complementary shapes, and are connected by a preferably separable mechanical connection.

Alternatively to an electronic watch such as the watch 10, a body monitoring system can comprise, as a wearable device, a watch without a screen (the term “tracker” is then commonly used), or else a bracelet electronics with or without electronic display elements. A tracker typically provides sports data and/or vertical positioning data (for example for the elderly) and/or GPS positioning data (for example for monitoring young children) and/or medical variable analysis data.

More generally, a body monitoring system can comprise any wearable device that can be positioned in regular contact with the skin.

Advantageously, the watch 10 further comprises a wireless communication interface, for example via a 3G and/or 4G and/or 5G and/or Wi-Fi and/or Bluetooth and/or NFC and/or DECT type telecommunication network.

Advantageously, the watch can comprise a luminous indicator such as a diode, which can be used to signal the end of a sensor preparation operation.

A wearable device such as the watch 10, equipped with a sensor 30, constitutes an example of a body monitoring system allowing to monitor the evolution of an individual's glycaemia.

### Micro-Needle Sensor

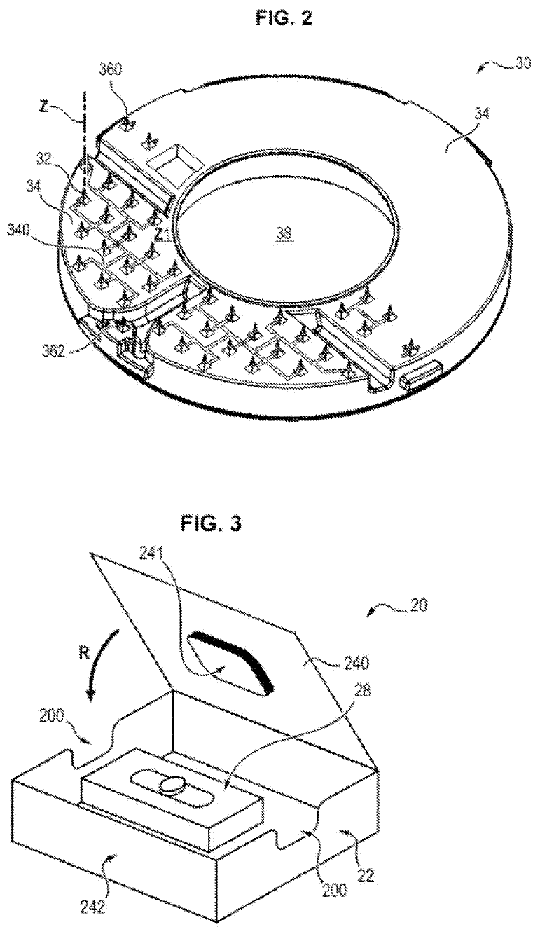

FIG. 2 illustrates a sensor 30 usable within the kit 1, according to an example.

The sensor 30 is a needle sensor designed to provide a measurement of electric current within the interstitial liquid of the wearer of the watch 10. The needles 32 are advantageously “micro-needles”. The sensor 30 preferably includes between four and fifty micro-needles. Note that a needle sensor for a body monitoring system can have any number of needles, starting from a single needle.

The term “micro-needle” means a needle having a short length, preferably between 10 micrometers and 1 millimeter, more preferably between 300 micrometers and 800 micrometers.

In the following, the micro-needles are referred to as “needles” to facilitate reading. However, needles other than micro-needles can also be used as an alternative.

The length of the needles 32 is thus sufficiently reduced to avoid contact with a nerve of the user, to limit the pain caused by wearing the watch 10.

Each needle here has a pyramidal shape. The needles 32 are arranged to form a network of needles.

In the present example, each needle 32 comprises on its surface at least one chemical or biochemical material capable of reacting with the body analyte of which it is desired to obtain a measurement (that is to say here glucose). A material capable of reacting with the body analyte is for example an enzyme capable of oxidizing the body analyte.

In an alternative example, each needle 32 comprises an internal cavity located at the rear of the tip, and the chemical or biochemical material capable of reacting with the analyte is located in this internal cavity.

In another alternative example, the sensor 30 may comprise cavities located at the rear of the needles 32, and/or inside the needles 32. For example, one or more needles 32 may comprise an open channel. The cavities comprise the chemical or biochemical material capable of reacting with the analyte. The needles 32 are then capable of bringing the body fluid up to said cavities.

The sensor 30 illustrated in FIG. 2 comprises, in addition to the needles 32, a substrate 34 provided with a plurality of metal tracks 340, a working conductivity electrode 360, a reference conductivity electrode 362 and a central opening 38.

During use of the sensor 30 to perform a measurement, a voltage is generated between several needles. At least a part of the needles 32 of the sensor 30 are at least partially immersed in the interstitial liquid. The chemical or biochemical material present on the surface of the needles 32 reacts with the glucose of the interstitial liquid.

The sensor 30 thus provides a measurement of electric current, representative of the concentration of glucose in the interstitial liquid.

The substrate 34 and the needles 32 are preferably arranged on a single face of the sensor 30, which is the face oriented upwards according to the orientation of FIG. 2. This upper face is intended to be arranged facing the skin of the user.

Each needle extends from the upper face in a direction Z, from its base to its tip. The direction Z is preferably orthogonal to a plane of the upper face.

When the sensor 30 is placed under the bearing means 24 to be prepared, this upper face is arranged facing the pouch 26. A displacement of the bearing means toward the bearing position causes the pouch 26 to be pierced by the needles 32.

The central opening 38 is circular in shape here. The sensor 30 thus has, in the present example, a generally annular shape.

For a detailed example of the structure of the sensor 30, reference may be made to the international application published under the number WO 2020/025822 and in particular to the description relating to FIGS. 1 and 2 of this document.

Preferably, the sensor 30 is integrated into a removable capsule. The removable capsule is designed to be admitted into the wearable device, here in the case 12 of the watch 10. The capsule can be coupled to the case 12, by mechanical coupling. The capsule is inserted into the back of the case 12. The case 12 and the capsule can have complementary shapes, which limits the force necessary for the correct insertion of the capsule against the case.

A support of the sensor 30 (for example the removable capsule) preferably comprises a patch (not shown) attachable to the skin of the user. The patch has an adhesive layer, or is itself formed from an adhesive material. The patch therefore allows the capsule to be attached to the skin of the wearer, and helps maintain the needles 32 in the interstitial liquid. The patch has for example an annular shape, and covers the capsule.

The sensor 30 is here provided to be controlled by the processing unit 14 of the watch 10. In a possible variant, the sensor 30 includes a memory, configured among other things to record resetting information. The resetting information can then be recorded in the sensor 30 after transmission by a processing unit integrated into the watch 10, during a method for calibrating the sensor 30.

### Example 1—Sensor Preparation Box

According to a first exemplary embodiment, at least part of the bearing means 24 of the sensor preparation assembly is integrated into a box. FIG. 3 illustrates a sensor preparation assembly according to this first example, comprising a box 20.

The box 20 comprises a cover 240 forming the upper face (according to the orientation of FIG. 3), and comprises side faces 22 and a lower face 242.

The box 20 here is generally parallelepipedic in shape. Preferably, the pouch 26 and the receptacle 28 are received in an internal space formed inside the box 20. When the sensor 30 is placed in front of the pouch 26 for its preparation, the sensor 30 is also received in the internal space of the box 20.

The receptacle 28 is either arranged against the lower face 242, or fixed to said face. In either case, the receptacle 28 is preferably received in a central part of the internal space of the box 20.

The cover 240 is integrated into the bearing means 24. In a simple exemplary embodiment, an attachment edge of the cover 240 is fixed to a side face 22. The other edges are free, to allow the cover 240 to pivot according to the direction R.

In a possible variant, at least one side face 22 comprises, on an upper edge left free by the cover 240, a housing 200 for placing a wearable device. The housing 200 is for example a concavity formed by removal of material. A part of the wearable device—for example the case 12 of the watch 10—can then be stably interposed between the cover 240 and the pouch 26.

If the desired wearable device is a watch, it is particularly advantageous for the housing 200 to be complementary with the bracelet 13 of the watch. The bracelet 13 is placed inside the housing 200, in a position transverse to the plane of the housing 200.

Preferably, the box 20 comprises two housings 200 placed face to face on two opposite side faces 22, these faces being located on the sides of the attachment edge of the cover 240. An advantage is to stabilize the bracelet 13 on both sides of the case 12.

The housing can also be provided to stabilize the receptacle 28 on which the pouch 26 is arranged.

A wedge (not shown) can be provided in the internal space of the box 20, and/or in the internal space of the housing 200, for example to ensure that the wearable device remains in place after its insertion into the box.

The cover 240 advantageously comprises a window 241. The window 241 is formed from a transparent or semi-transparent material. The window 241 advantageously has dimensions similar to or slightly larger than the dimensions of the screen 16, and extends opposite the screen 16 when the watch 10 is received in the box 20.

Thus, information displayed on the screen 16 remains visible when the watch 10 is placed in the closed box 20.

In another possible variant, the housings 200 can be omitted. Indeed, it is not necessary to use as bearing means a watch part 10 or a part of any other wearable device. It is possible to contact the needles 32 and the pouch 26, by pressing the cover 24 directly on the sensor 30.

The lower face 242 is, in turn, preferably fixed and flat. The box 20 is thus easily positioned on a flat surface. The box 20 is preferably formed of a light material that is easy to shape, such as a polymer.

The sensor preparation assembly shown in FIG. 3 is very easy and quick to use. The user does not need to directly handle the needles 32 of the sensor 30. For example, he removes the watch 10 and places the sensor which must be prepared inside the watch 10, then he closes the cover 240 on the watch 10 and on the sensor. There is no risk of injury to the user.

Another advantage of the sensor preparation assembly of FIG. 3 is that the box 20 does not need to be electrically powered. In the example illustrated, the box 20 does not embed any electronic component.

In a possible variant, the box 20 can however be supplied with electric current, for example if the box 20 comprises a luminous indicator such as a diode to inform the user of a duration for maintaining the bearing position.

Furthermore, the box 20 forms a packaging device whose storage and transport are easy.

In a possible variant, the cover 240 of the box 20 can be omitted. A box open on the top is thus obtained. It is then possible to use as bearing means either a part of a wearable device, or a mechanical actuator of the wearable device, or the hand of the user himself, or any other way.

### Example 2—Electronic Watch Charging Station

According to a second exemplary embodiment, at least part of the bearing means of the sensor preparation assembly is integrated into a charging station.

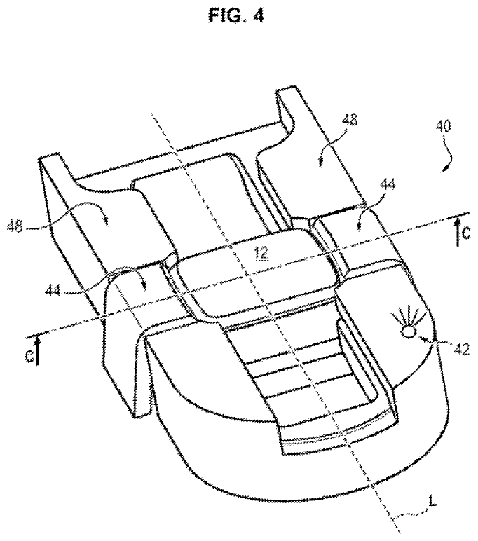

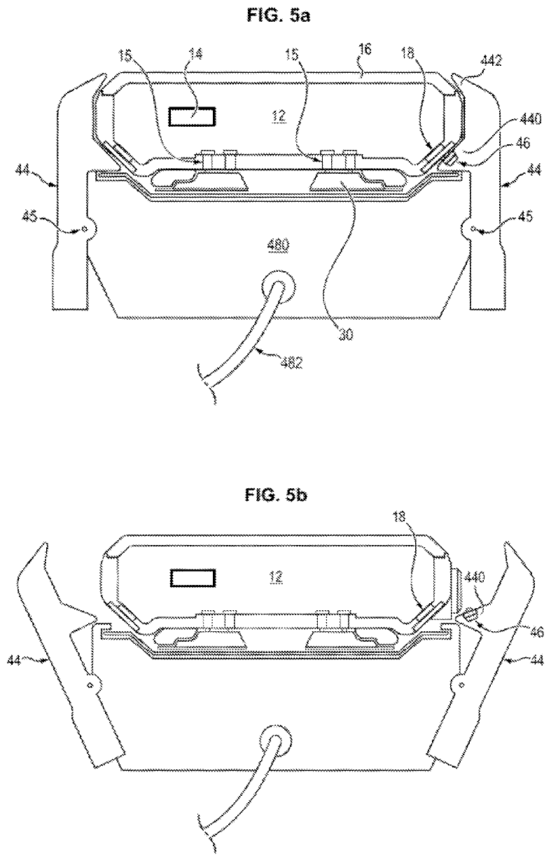

A charging station 40 usable for the preparation of a needle sensor, for example for the preparation of the sensor 30, is illustrated in FIG. 4, FIG. 5a and FIG. 5b.

FIG. 4 is a top view of the charging station 40 in which the case 12 of the watch 10 is inserted.

FIGS. 5a and 5b are sectional views along the section plane C-C of FIG. 4.

In these figures, the case 12 is coupled with a sensor 30. The station 40 constitutes a “dock” complementary with the case 12.

A receiving space of the case is formed on top of a central area of the station 40. Preferably, the receiving space of the case is formed between two mechanical clips 44 of the station 40.

Here, the receiving space of the case is adapted to receive the case 12.

The station 40 further comprises at least one mechanical clip for pressing the case 12, here two side mechanical clips 44. The case 12 constitutes here, in complementarity with the mechanical clips 44, means for bearing the sensor 30 against the pouch 26.

The mechanical clips 44 are movable between a separated position and a close position. The position illustrated in FIG. 4 and FIG. 5a is a close position. The position illustrated in FIG. 5b is a separated position.

Depending on the close or separated position of the mechanical clips 44, the case 12 presses or does not press the sensor 30 in the direction of the pouch 26.

Thus, the station 40 plays the role of preparation station for the sensor 30.

The station 40 is designed to receive the receptacle 28 below the case 12, and the pouch 26 below the sensor 30. For more readability of the figures, the area for receiving the receptacle 28 and the pouch 26 is not shown in FIGS. 4, 5a and 5b.

The receptacle 28 and the pouch 26 are removable with respect to the station 40. Alternatively, the receptacle 28 is fixed to the station 40. Still alternatively, a surface of the station 40 acts as a receptacle; thus, the pouch 26 is arranged directly on the station 40 and is not carried by a structural element separate from the station.

Alternatively, the mechanical clips 44 can be omitted. A cover can be used to bear the case 12 against the sensor 30, or the user can be asked to directly bear the case.

It may be required that the user positions the case 12 in the receiving space, and bears on the case 12 to press the sensor.

In a case where the preparation of the sensor includes a pre-wetting and a pre-calibration, the needles 32 can be pressed inside the pouch 26 for a duration comprised between 1 minute and 120 minutes to carry out the pre-wetting. The indicator 42, or, alternatively, an indicator present on the wearable device, can be activated to notify the end of the pre-wetting to the user. The pre-calibration of the sensor 30 is carried out at the end of the pre-wetting.

Alternatively, the pre-calibration is carried out before the pre-wetting of the needles.

In either case, the duration required for pre-calibration (for example comprised between 10 seconds and 2 minutes) is generally much shorter than the time required for pre-wetting (for example between 1 minute and 120 minutes).

Returning to FIG. 4, the mechanical clips 44 and the case 12 are aligned on a median plane of the station 40. A longitudinal axis L of the station 40, shown in dotted line in FIG. 4, is orthogonal to the median plane. Preferably, the side edges of each of the mechanical clips 44 are aligned with the side edges of the case 12.

In their close position, each of the mechanical clips 44 is positioned longitudinally between two blocks 48 of the station 40. The mechanical clips 44 are movable relative to the blocks 48, so as to move close or apart from each other.

Preferably, a channel is made in the volume of the blocks 48 (here along the longitudinal axis L) to allow the user to easily insert and grasp the case 12 when the mechanical clips 44 are in a separated position.

The station 40 advantageously comprises an actuator (not shown), preferably an electric actuator, for controlling a separation or a rapprochement of the mechanical clips 44. The actuator is for example a push button that the user can actuate to switch the position of the mechanical clips 44 between the close and separated positions.

According to another option, the mechanical clips 44 are switched between the close position and the separated position by light pressure from the user. The mechanical clips 44 can “click” and change position.

Preferably, the station 40 further comprises an indicator 42 to inform a user of a duration for maintaining the bearing position of the case 12 and of the mechanical clips 44. The indicator 42 is placed on an upper surface of the station 40. The indicator 42 here is a diode.

FIGS. 5a and 5b further illustrate respectively the close position and the separated position of the mechanical clips 44.

As can be seen in FIGS. 5a and 5b, a height of each of the mechanical clips 44 is equal to a combined height of the case 12 and of a lower portion 480 of the station 40.

Each mechanical clip 44 is pivotally mounted on itself, around a respective axis 45. Here, the two axes 45 extend orthogonally to the cutting plane C-C. Thus, the mechanical clips 44 are movable in rotation along the cutting plane C-C.

Preferably, the two mechanical clips 44 are symmetrical with respect to a longitudinal plane of the charging station, that is to say here with respect to the plane orthogonal to the cutting plane C-C passing through the longitudinal axis L.

Each mechanical clip 44 forms, on an upper end, a concavity adapted to receive one side of the case 12.

In this example, each mechanical clip 44 comprises a tip 442 at a distal position and comprises a tooth 440 at a medial position, the tip 442 and the tooth 440 extending inward. A receiving concavity is formed between the tip 442 and the tooth 440.

In the close position of FIG. 5a, the two mechanical clips 44 are in contact with the case 12 and carry out a bearing in Z on the case 12. The sensor 30, placed under the case 12, is pressed in the direction of the pouch 26 positioned below the sensor. The tips 442 prevent inadvertent extraction of the case 12.

In an alternative embodiment, the mechanical clips can be configured to carry out a bearing in Z directly on the sensor 30 itself. In this case, it is not necessary to insert the wearable device into the station 40 during a pre-calibration of the sensor 30.

In the separated position of FIG. 5b, the two mechanical clips 44 are not in contact with the case 12. The case 12 can be extracted from the station 40.

Optionally and advantageously, at least one mechanical clip 44 (here the right mechanical clip) comprises an electrical contact 46, arranged on a contact surface with the case 12. Preferably, the electrical contact 46 is arranged on a surface of lateral contact with the box. The electrical contact 46 is here arranged on the tooth 440.

The station 40 is configured to supply electric current to a battery (not shown) of the watch 10, via the electric contact 46. The watch 10 comprises, as such, a watch contact 18 intended to cooperate with the electrical contact 46. In the close position of FIG. 5a, the watch contact 18 is against the electrical contact 46 of the station 40.

Moreover, sensor contacts 15 are provided in the case 12, in order to electrically connect the electronics of the case 12 and the sensor 30. The sensor contacts 15 allow the case 12 not only to supply electric current to the sensor 30, but also to exchange data with the sensor 30. It will be noted that, alternatively or in combination, the case 12 and the sensor 30 can each comprise a wireless communication interface, such as an NFC interface.

Alternatively, electrical contacts separate and mechanically independent of the mechanical clips 44 are provided in the charging station, for charging the battery.

The station 40 is itself supplied with current, preferably by a power cord 482, a first end of which comprises an electrical outlet and the second end of which is fixed to the lower portion 480.

The station 40 is thus preferably both a sensor preparation station and an electric watch charging station.

The screen 16 of the watch 10 is directed toward the outside of the station 40. Thus, information indicative of the charging of the battery and/or of the sensor preparation operations can be displayed on the screen 16 during the use of the station 40.

The station 40 here has a U-shape when the station is seen from above. Alternatively, the station 40 has a parallelepipedic shape, or any other shape.

The functions of electrically charging a battery of the wearable device on the one hand, and of preparing the needle sensor of the wearable device (in particular of calibrating said sensor) on the other hand, can thus be fulfilled by the same station 40. This reduces the number of items of equipment that the user must group together and keep in order to operate the body monitoring system.

Alternatively, the station 40 performs the preparation of the sensor 30 completely autonomously, without requesting the watch 10. The station 40 then embeds the electronics necessary to implement, for example, a pre-calibration measurement and a resetting as described below. A possible option is to press the needles 32 directly inside the pouch 26 by pressing the sensor 30 via the mechanical clips 44, or via any other mechanical actuator.

In an alternative example (not illustrated in the appended figures), the sensor preparation assembly comprises a receptacle itself equipped with part of the bearing means. For example, the sensor bearing means comprise the casing 12 as well as side edges for clipping the receptacle 28, capable of blocking the casing 12 in the bearing position against the sensor 30.

An advantage of this last example is to omit the need for an additional component, such as a box or a preparation station. In addition, the shape of the case 12 being simple, it is easy to manufacture a receptacle comprising clipping edges complementary with the box.

### Sensor Preparation Method—Example of a Sensor Pre-Calibration

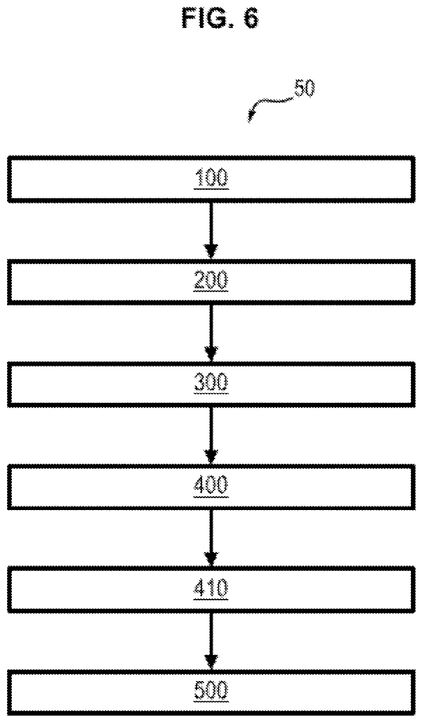

FIG. 6 illustrates the steps of a method 50 for preparing a needle sensor for a body monitoring system, according to an example in which the preparation of the sensor comprises a “pre-calibration” of the sensor.

“Pre-calibration” means a calibration operation of the measurement provided by the needle sensor, which occurs at the start of the use cycle of the sensor.

The pre-calibration of the sensor corrects any drift in the electrical current measurement provided by needles 32 of the sensor. The needles 32 are immersed in a volume of a calibration solution contained in the pouch 26. The concentration of body analyte (here in glucose) of the calibration solution being known, the value of the expected electrical current measurement is also known. A correction of the electric current measurement, or “drift correction”, is thus performed according to the value of the expected electric current measurement.

The pre-calibration is advantageously carried out immediately before the insertion of the sensor (preferably new) in a wearable device. In the present example, the sensor 30 is pre-calibrated before it is inserted into the watch 10.

The sensor 30 may or may not have undergone a “factory calibration” prior to the pre-calibration. It is reminded that a factory calibration comprises, after the production of a batch of needle sensors, the inscription of an alphanumeric code on a packaging of the sensor or the integration of a chain of characters representative of said code in a memory of the sensor. The alphanumeric code can be recognized by an electronic apparatus having an abacus, where the alphanumeric code is associated with a body analyte concentration reference value.

It should be noted that a method for preparing the needle sensor does not necessarily include a calibration of a measurement provided by the sensor. The preparation of the sensor may include only a wetting of the needle, in particular a “pre-wetting” to accelerate the subsequent convergence of the measurement provided by the sensor.

Sensor preparation may also include a combination of pre-wetting and pre-calibration. An advantage of the sensor preparation assemblies described above is to allow simultaneous wetting and calibration, by means of the immersion of at least one needle 32 in the volume of the pouch 26.

At an optional step 100, the peelable film possibly present on the receptacle 28 is removed.

Thus, the pouch 26 received in the receptacle is exposed to the open air. Insofar as the receptacle 28 preferably also comprises the sensor 30 before removal of the peelable film, the sensor 30 is preferably also in the open air after removal of the film. Step 100 can be implemented when the receptacle 28 is already arranged facing the bearing means 24, or beforehand.

Next, the sensor 30 is placed in a step 200 in a position that is interposed between the bearing means 24 and the pouch 26. In the example of the sensor in FIG. 2, the upper face carrying the needles is arranged opposite the pouch containing the preparation solution. The needles 32 then face the pouch 26.

In a preferred case, the sensor 30 is integrated into an interchangeable removable capsule. The removable capsule is placed in the wearable device, here on the back of the case 12, prior to step 200.

By way of example, the placement of the sensor can be carried out as follows:

if the sensor preparation assembly is in accordance with FIG. 3, the cover 24 is open and the sensor 30 is placed above the receptacle 28 in the box 20;

if the sensor preparation assembly is in accordance with FIG. 4, the mechanical clips 44 are placed in the separated position, the receptacle 28 is placed in the reception space and the sensor 30 is placed above the receptacle 28.

Then, the bearing means are placed in the bearing position, so that at least one needle, preferably a plurality of needles 32, pierces the pouch 26 and is at least partially immersed in the calibration solution. Preferably, the needles are completely immersed in the solution.

It is recalled that the bearing means are for example formed of the cover 24 in combination or not with the case of the watch, or of the mechanical clips 44 in combination or not with the case of the watch.

Preferably, the bearing means are maintained in their bearing position for a duration comprised between 10 seconds and 2 hours. The immersion duration of the needles 32 is thus sufficient to implement a pre-calibration measurement and a resetting, according to the steps 300 and 410 described below, and/or to implement a pre-wetting of the needles 32.

The duration of maintaining the bearing means in the bearing position is even more preferably comprised between 1 minute and 120 minutes, in particular in the case of a pre-wetting.

Alternatively, the duration of maintaining the bearing means is advantageously comprised between 10 seconds and 2 minutes in the case of a pre-calibration carried out alone.

It will be noted that the watch 10 is not necessarily requested for the steps 200 and 300. For example, a sensor preparation assembly in accordance with FIG. 3 can include a cover 24 capable of pressing the needles 32 inside the pouch 26, without the watch 10. Thus, even if the duration of maintaining the bearing means is long, the user can continue his normal use of the watch 10 during the preparation of the sensor.

To carry out the pre-calibration of the sensor 30, the method 50 comprises additional steps 300 to 410.

In step 300, the sensor 30 is controlled to acquire a measurement of the concentration of a body analyte, here the concentration of glucose, of the preparation solution contained in the pouch 26.

The sensor 30 is preferably controlled by the processing unit 14 contained in the case 12 of the watch 10. Alternatively, the sensor 30 is controlled by a processing unit of another electronic apparatus, for example a processing unit of a calibration station (such as station 40 described above).

The measurement acquired can be qualified as a “pre-calibration measurement”.

In step 400, the pre-calibration measurement is transmitted to the processing unit of the electronic apparatus. In the present example, the sensor 30 is already in place on the case 12 and can exchange data with the case 12 during the measurement.

Alternatively, the pre-calibration measurement can be saved in an electronic memory embedded in the sensor 30, for later reading by the wearable device. Still alternatively, the pre-calibration measurement is saved in a memory of another apparatus (for example a memory of the station 40), then transmitted to the wearable device by wired or wireless communication.

In step 410, a registration signal is calculated. The resetting signal is preferably obtained from the pre-calibration measurement, and from the body analyte concentration reference value in the calibration solution.

For example, if the glucose concentration reference value is 10 millimoles per liter, and if the electrical current measurement acquired by the sensor 30 in step 300 corresponds to a concentration of 9 millimoles per liter, then the resetting signal comprises code instructions for adjusting the measurement until it corresponds to 10 millimoles per liter.

In other words, the glucose concentration in the calibration solution of the pouch 26 is used as a reference to readjust the measurement provided by the sensor 30.

The resetting signal is saved, so that the resetting is carried out automatically during the subsequent measurements supplied by the sensor 30.

Preferably, the resetting signal is recorded in a memory of the watch 10, in association with a reference of the capsule containing the sensor 30. The reference of the capsule is for example an alphanumeric code assigned to the capsule, at the end of the production of the capsule.

Once the resetting signal has been calculated and saved by the watch, a visual indicator on the watch can be lit to indicate the end of the pre-calibration operation. In particular, the preparation assembly illustrated in FIG. 3 comprises a transparent or semi-transparent window 241 which allows the user to see the end of the pre-calibration operation while the cover 24 is closed.

Alternatively, the resetting signal is recorded in a memory of the sensor.

At the end of step 410, the bearing position of the bearing means is released and the sensor 30 is released. The sensor 30 can then be moved and integrated into a wearable device during an optional step 500, if its integration into the wearable device has not already been carried out beforehand.

At the end of the preparation of the sensor (comprising for example the pre-calibration and/or the pre-wetting), the pouch 26 is preferably discarded. A new pouch 26 is preferably used for any new iteration of the sensor preparation method 50, to ensure perfect sterility of the sensor 30.

Such a pre-calibration method is particularly useful when replacing the interchangeable removable capsule containing the sensor. A new removable capsule comprising a non-pre-calibrated sensor 30 is, for example, inserted into the back of the case 12. Then, a sensor preparation assembly according to any one of the examples described above is used for in particular pre-wetting and pre-calibrating the sensor 30. An advantage is that the new removable capsule is already inserted into the watch 10 at the end of the preparation of the sensor.

The method 50 thus allows to correct the possible drift of the electrochemical measurement provided by a needle sensor for a body monitoring system. The resetting of the sensor measurement is carried out in a simple, fast and reliable manner. It is advantageous to carry out, simultaneously or sequentially, the resetting of the measurement and the pre-wetting of the needles of the sensor, which is made possible by the sensor preparation assemblies described above.

Moreover, the resetting of the measurement of the sensor can be carried out simultaneously with the charging of a battery of the wearable device, as seen above.

The pre-calibration of the sensor increases the reliability of the measurement provided by the sensor. In the present example, the pre-calibration of the sensor, possibly associated with a “factory calibration” at the end of production, ensures satisfactory accuracy of the glycaemia measurement throughout the cycle of use of the wearable device.

1. A sensor preparation assembly for a body monitoring system, the sensor comprising at least one needle, the preparation assembly comprising:
a pouch comprising a volume of a preparation solution, the pouch being configured to be positioned under the sensor and being configured to be pierced by the needle,
a receptacle on which the pouch is arranged,
bearing means that are movable in relation to the receptacle toward a bearing position, the bearing means in the bearing position being configured to cause the sensor to pierce the pouch.

2. The assembly according to claim 1, wherein the preparation solution is a calibration solution having a concentration by volume of a body analyte equal to a predetermined reference value.

3. The assembly according to claim 2, wherein the body analyte is glucose, and wherein the predetermined reference value is comprised between 2 millimoles of glucose per liter and 20 millimoles of glucose per liter, preferably between 6 millimoles of glucose per liter and 10 millimoles of glucose per liter.

4. The assembly according to claim 1, wherein the preparation solution is a pre-wetting solution.

5. The assembly according to claim 1, further comprising an electronic apparatus, the electronic apparatus preferably being configured to control a concentration measurement of a body analyte via the sensor and/or configured to generate an electric current.

6. The assembly according to claim 5, wherein the electronic apparatus comprises a wearable device, preferably an electronic watch or bracelet or a tracker.

7. The assembly according to claim 5, wherein the electronic apparatus comprises at least one processing unit and a memory, configured to record and manage information on the resetting of a measurement of the body analyte concentration.

8. The assembly according to claim 5, wherein part of the electronic apparatus forms the bearing means or is part of the bearing means.

9. The assembly according to claim 5, wherein the bearing means comprise a cover.

10. The assembly according to claim 1, comprising a box, at least part of the bearing means being integrated into the box, the pouch and the receptacle preferably being received in the box.

11. The assembly according to claim 10, wherein the box comprises at least one housing configured to stably arrange the receptacle or a wearable device in the box.

12. The assembly according to claim 1, wherein the bearing means comprise at least one mechanical clip configured to perform a bearing in Z on the sensor, or on part of an electronic apparatus.

13. The assembly according to claim 1, further comprising a peelable film, the peelable film forming with the receptacle an enclosure which encloses the pouch and/or the sensor.

14. The assembly according to claim 1, further comprising a preparation station configured to receive the pouch, the preparation station preferably comprising an indicator to inform a user of a duration of maintaining the bearing position.

15. The assembly according to claim 14, wherein the preparation station further comprises a bearing cover and/or at least two mechanical clips movable relative to each other in a separation direction.

16. A sensor kit including a sensor preparation assembly according to claim 1, the sensor kit further including a sensor comprising at least one needle,
the sensor being configured so that, in the bearing position of the bearing means, the needle is partially or totally immersed in the preparation solution.

17. The kit according to claim 16, wherein the sensor comprises a processing unit and a memory, configured to record and manage information on the resetting of a measurement of the body analyte concentration.

18. A method for preparing a sensor of a body monitoring system, the method being implemented using a sensor kit according to claim 16, the method comprising a step of placing the sensor in a position that is interposed between the bearing means and the pouch, the bearing means being placed in the bearing position so that the needle of the sensor pierces the pouch.

19. The preparation method according to claim 18, wherein the bearing means are integrated into an electronic apparatus which further comprises a processing unit.

20. The preparation method according to claim 19, the method comprising additional steps of:
acquiring by the sensor a measurement of the concentration of a body analyte in the solution for preparing the pouch,
transmitting the measurement to the processing unit,
calculating a resetting signal according to the measurement, so as to calibrate the sensor.

21. The preparation method according to claim 18, wherein a wearable device is interposed between the bearing means and the sensor, the bearing means (24) preferably comprising a cover which presses on the wearable device.

22. The preparation method according to claim 18, wherein the bearing means are maintained in the bearing position for a duration comprised between 10 seconds and 2 hours, in order to press the sensor.
